(19) United States
(12) Patent Application Publication
(10) Pub. No.: US 2004/0254466 A1
(43) Pub. Date: Dec. 16, 2004

(54) Apparatus and method for real time three-dimensional ultrasound imaging

(76) Inventors: James Boner, Lynnwood, WA (US); Xiang-Ning Li, Mill Creek, WA (US); Paul Detmer, Seattle, WA (US)

Correspondence Address: ATL ULTRASOUND, P.O. BOX 3003, 22100 BOTHELL EVERETT HIGHWAY, BOTHELL, WA, 98041-3003, US

(21) Appl. No.: 10/825,933

(22) Filed: Apr. 16, 2004

Related U.S. Application Data
(60) Provisional application No. 60/479,215, filed on Jun. 16, 2003.

(51) Int. Cl. A61B 8/00

(52) U.S. Cl. 600/447

(57) ABSTRACT

The present invention is directed to ultrasound systems for three-dimensional ultrasound imaging data in real time. In one embodiment, the system includes a processing system coupled to an ultrasound scan head 40 that includes an ultrasound transducer array 30 coupled to a positional actuator 32 having a driven member that rotates about a first axis to pivot the array about a second axis substantially perpendicular to the first axis. In another embodiment, an ultrasound scan head 40 includes a positional actuator 42 rotatable about a first axis and coupled to a pivot member that supports an array that rotates about a second rotational axis substantially perpendicular to the first axis. In yet another embodiment, a method for three-dimensional imaging includes controlling the rotation of a driven member over a predetermined rotational pattern to provide approximately constant rotation of the array; and acquiring ultrasound data along a plurality of mutually spaced-apart scan lines.

This invention claims the benefit of Provisional U.S. patent application Ser. No. 60/479,215, filed Jun. 16, 2003.

The present invention relates generally to ultrasound diagnostic systems that use ultrasonic transducers to produce ultrasonic echoes from the interior of a body, and more particularly to ultrasound diagnostic systems capable of acquiring three-dimensional ultrasound imaging data in real time.

Ultrasonic diagnostic imaging systems are widely used for a variety of medical diagnostic tasks where it is desired to visualize selected internal anatomical regions of a patient. Diagnostic images are obtained from these systems by placing an ultrasound scan head in contact with an exterior portion of the patient's body and transmitting ultrasonic signals into the body of the patient. Ultrasonic echoes reflected from internal organs or other tissues within the body are then received by the scan head and converted into electrical signals that are processed by an ultrasound system so that a visual image of the internal anatomical portion under examination may be formed. The visual image may then be viewed on a display device generally associated with the system.

Conventional ultrasound scan heads generally include a linear array of transducer elements that may be separately excited by the system so that a two-dimensional visual representation of the internal anatomy of the patient is produced. By manually positioning the conventional scan head relative to an anatomical region, a series of two-dimensional images are generated, which may be used to approximate a three-dimensional view of the anatomical region. In practice, however, numerous difficulties exist. Imprecise positioning of the scan head may result in a misleading estimation of the underlying anatomy and functions. Geometrical limitations of the scan head itself may further lead to two-dimensional images that at least partially obscure important anatomical details. Moreover, once the two-dimensional images are acquired, they may be difficult to interpret, since several two-dimensional images must be mentally integrated in order to form an approximation of a three-dimensional image of the anatomical structure. The variability thus introduced may lead to an incorrect diagnosis.

Accordingly, it is desirable to form a three-dimensional image of a region of interest in the body directly, so that the foregoing difficulties are avoided. To generate a three-dimensional image, the array is generally swept across an external area of the body near the internal region of interest by a translation mechanism, and a set of two-dimensional image data sets is accumulated along discrete scan lines that are mutually and laterally spaced apart. The resulting set of images are stored in a memory within the imaging system that processes the stored two-dimensional data sets and constructs a three-dimensional static representation of the internal region of interest. If the scan head is repetitively swept across the external area of the body, sets of static images may be repetitively generated that may be further processed to produce a real time volumetric image of the internal region of interest. Three-dimensional representations of an anatomical region in real time affords significant benefits. For example, three-dimensional real time imaging permits fetal motion as well as quantitative estimates of fetal development may be obtained. Additionally, cardiac motion and volume may also be conveniently obtained that may be useful in the diagnosis and treatment of various cardiac diseases.

In order to form accurate three dimensional representations of an internal anatomical portion in real time, the transducer array within the scan head must be rotated or translated by a mechanism within the scan head so that the array is positioned at predetermined and accurately controlled positional intervals as the array scans across the anatomical region of interest. In particular, the positional interval between successive scan lines must be accurately controlled. If the positional interval is subject to variation, which may result from excessive clearances in the mechanical elements of the mechanism, a three-dimensional image having significant geometrical distortion may result.

Various scan head devices have been developed that permit an array to be scanned across an area at controlled intervals. For example, U.S. Pat. No. 5,460,179 to Okunuki, et al. discloses an ultrasonic transducer assembly having an ultrasonic array positioned on an internally mounted rocking assembly that is coupled to a rotating motor drive by a flexible drive belt. The angular intervals between two-dimensional scans are recorded by a rotational encoder positioned on the motor shaft. Accordingly, the angular position of the array may be inferred from the rotational position of the motor drive shaft. One significant shortcoming present in this approach is that drive belt wear and/or drive belt stretching may cause significant positional errors to occur, thus causing distorted three-dimensional images.

Other prior art approaches have employed a mechanical linkage that directly couples the transducer array to a motor that positions the array. U.S. Pat. No. 4,913,155 to Dow, et al. discloses a linear motor that is coupled to a gimbal-mounted transducer by a linear connection member that transfers the linear motion of the motor to the gimbal to impart an oscillatory motion to the transducer. Although the disclosed device avoids the use of drive belts, or other similar elements that may introduce undesired relative movement between the array and the drive motor, other shortcomings nevertheless exist. For example, since the linear motor impulsively moves as it positions the transducer, vibrational motions may be generated by the device during an ultrasound examination that may be disturbing to a patient.

Still other prior art approaches avoid oscillation of the transducer array by imparting a constant rotational speed to the transducer array. For example, U.S. Pat. No. 5,159,931 to Pini discloses a scan head having a tubular housing that supports the transducer at one end of the housing. The transducer is further coupled to a drive motor that rotates the transducer about a longitudinal axis of the housing to permit the repetitive acquisition of ultrasound data. Since the ultrasound transducer emits and receives ultrasonic signals from the end of the housing, however, the device has a relatively small aperture size, thus limiting the lateral resolution of the device as the scanning depth is increased.

Accordingly, there is a need in ultrasound imaging for a ultrasound scan head that includes a mechanism for moving and positioning the scan head array that avoids the use of flexible drive members, or other components that may introduce significant positioning errors when employed in three-dimensional real time imaging. The mechanism should further avoid the generation of undesirable vibrational motions that may be disturbing to a patient during an ultrasound examination. Still further, the positioning mechanism should permit wide aperture operation, and be convenient to use.

The present invention is directed to ultrasound diagnostic systems that use ultrasonic transducers to produce ultrasonic echoes from the interior of a body, and more particularly to ultrasound diagnostic systems capable of acquiring three-dimensional ultrasound imaging data in real time. In one aspect, an ultrasound imaging system includes a processing system to generate ultrasound energy and to detect signals at ultrasound frequencies, the processing system being coupled to an ultrasound scan head that includes an ultrasound transducer array operatively coupled to a positional actuator having a driven member that rotates about a first axis to pivot the array about a second axis substantially perpendicular to the first axis.

In another aspect, an ultrasound scan head for ultrasound imaging includes an ultrasound transducer array having a plurality of transducer elements for transmitting acoustic energy in response to an applied electrical signal and transducing returned acoustic energy into electrical signals, a positional actuator configured to be rotated about a first rotational axis and coupled to a pivot member that supports the array, the pivot member being configured to rotate about a second rotational axis substantially perpendicular to the first axis, and a positional sensor coupled to the positional actuator to sense a rotational position associated with the positional actuator.

In yet another aspect, a method for three-dimensional imaging a portion of a body using a scan head having a driven member rotatable about a first axis and coupled to an ultrasound array rotatable about a second axis includes controlling the rotation of the driven member over a predetermined rotational interval to provide approximately constant rotation of the array; and acquiring ultrasound data along a plurality of mutually spaced-apart scan lines.

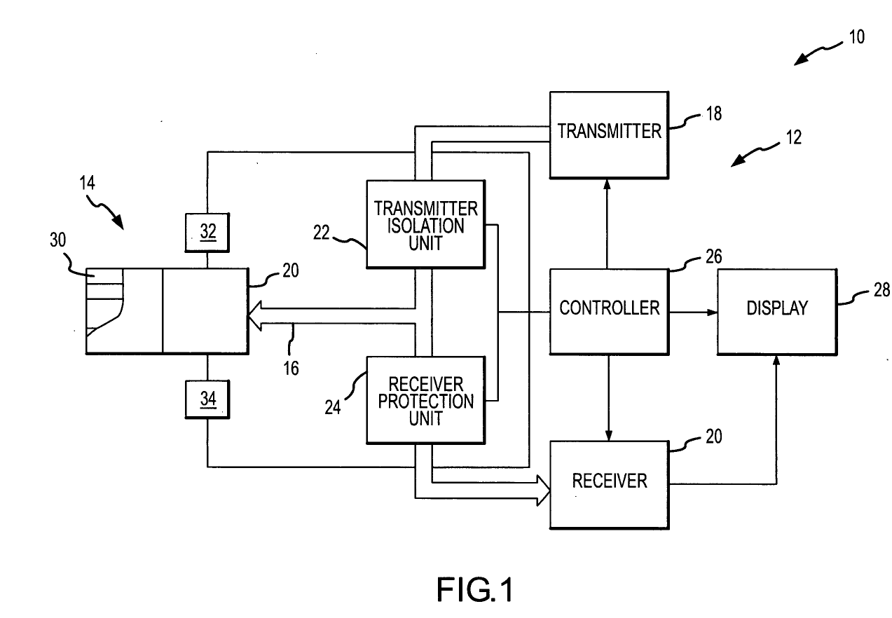

FIG. 1 is a functional block diagram of an ultrasound imaging system according to an embodiment of the invention.

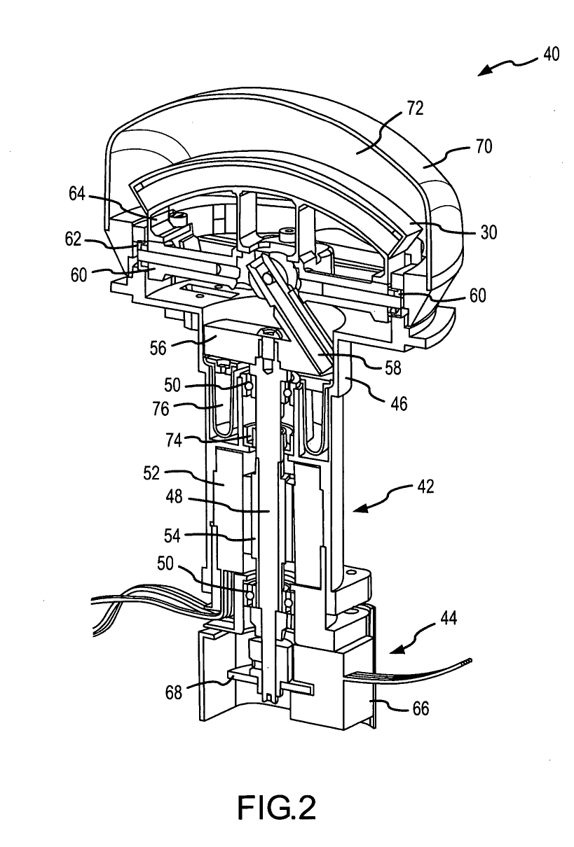

FIG. 2 is a cross sectional isometric view of a scan head according to another embodiment of the invention.

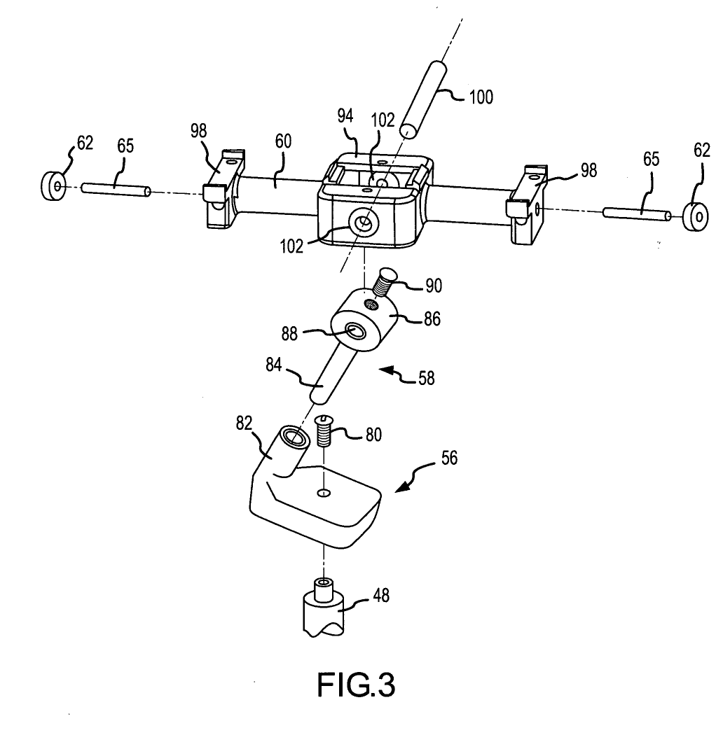

FIG. 3 is a partial isometric view of a scan head according to another embodiment of the invention.

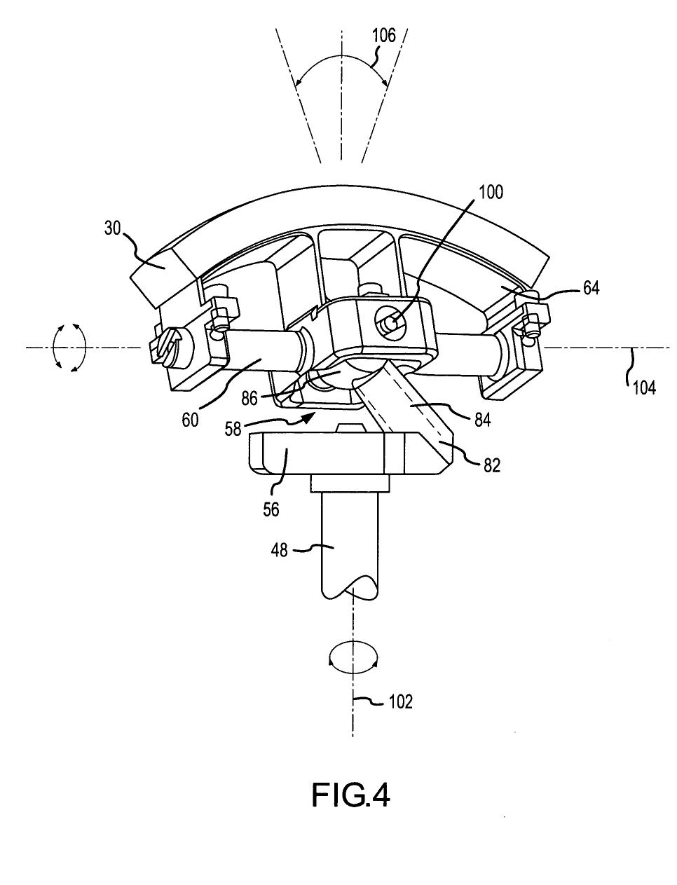

FIG. 4 is a partial side view of the scan head that shows scanning angle operating modes according to still another embodiment of the invention.

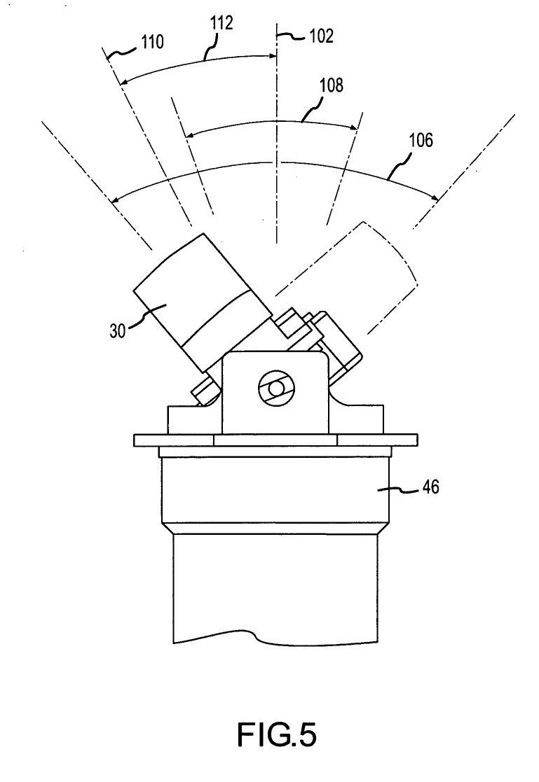

FIG. 5 is a graph that illustrates the angular position of a transducer assembly as it sweeps through a scanning angle.

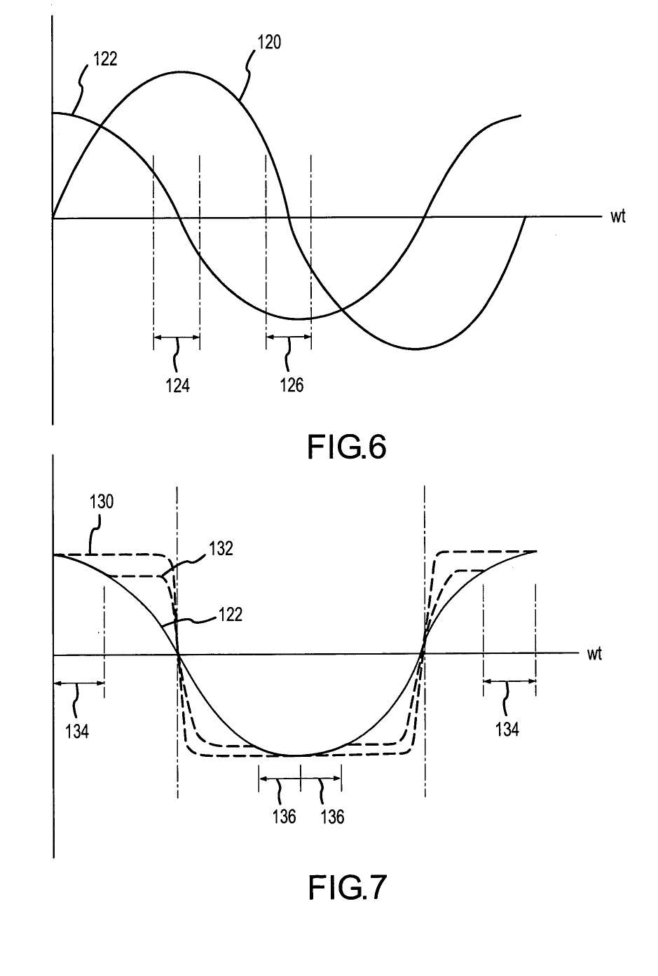

FIG. 6 is a graph that illustrates a method for controlling a scanning rate for a scan head according to yet another embodiment of the invention.

FIG. 7 is a graph that illustrates a method for controlling a scanning rate for a scan head according to yet another embodiment of the invention.

The present invention is generally directed to an ultrasound diagnostic systems that use ultrasonic transducers to produce ultrasonic echoes from the interior of a body, and more particularly to ultrasound diagnostic systems capable of acquiring three-dimensional ultrasound imaging data in real time. Many of the specific details of certain embodiments of the invention are set forth in the following description and in FIGS. 1 through 7 to provide a thorough understanding of such embodiments. One skilled in the art will understand, however, that the present invention may be practiced without several details described in the following description.

FIG. 1 is a functional block diagram of an ultrasound imaging system 10 according to an embodiment of the invention. The system 10 includes an ultrasound processor 12 that is coupled to a scan head 14 by a connecting cable 16. The ultrasonic processor 12 includes a transmitter 18 that generates signals at ultrasonic frequencies for emission by the scan head 14, and a receiver 20 to detect signals received by the scan head 14. In order to isolate the transmitter 18 from the scan head 14 while the receiver 20 is in operation, a transmitter isolation unit 22 decouples the transmitter 18 from the cable 16. Correspondingly, when the transmitter 18 is in operation, a receiver protection unit 24 decouples the receiver 20 from the cable 16. A controller 26 interacts with the transmitter 18, the receiver 20, the transmitter isolation unit 22 and the receiver protection unit 24 to coordinate the operation of these components. The controller 26 similarly interacts with a display system 28 to permit signals received by the processor 12 to be visually displayed.

The scan head 14 includes a transducer assembly 30 that is comprised of one or more piezoelectric elements that are configured to emit ultrasonic pulses in a desired direction when excited by signals generated by the transmitter 18, and to convert the reflected portions of the pulses into electrical signals that may be detected by the receiver 20. The transducer assembly 30 may include a one-dimensional array of transducer elements arranged in a planar, convex or even a concave arrangement of elements. In addition, the transducer assembly 30 may include other higher dimensional arrays of elements, such as a 1.5 or even a two-dimensional array.

Still referring to FIG. 1, the scan head 14 further includes a positional actuator 32 that is coupled to the transducer assembly 30 to position the transducer assembly 30 in a desired direction, and further to repetitively scan an anatomical region in the desired direction so that a real-time image of the region may be formed. The positional actuator 32 is coupled to the controller 26 through the cable 16 to transmit control inputs from the controller 26 to the actuator 32 so that the movement of the transducer assembly 30 may be controlled. The actuator 32 may be controlled, for example, by controlling a voltage or a current transferred to the actuator 32. Alternatively, the actuator 32 may be controlled by transferring a control signal from the controller 26 to a separate controller located within the scan head 14 that further controls a current or a voltage transferred to the actuator 32. The scan head 14 also includes a positional sensor 34 that is coupled to the transducer assembly 30. The positional sensor 34 determines the directional orientation of the transducer assembly 30 as it is moved by the positional actuator 32, and is similarly coupled to the controller 26 by the cable 16 to provide positional input signals to the controller 26.

FIG. 2 is a cross sectional isometric view of a scan head 40 according to another embodiment of the invention. The scan head 40 includes a positional actuator 42 that is mechanically coupled to a transducer assembly 30 and a positional sensor 44. The transducer assembly 30, the positional actuator 42 and the positional sensor 44 are positioned within a supporting structure 46. The positional actuator 42 includes a drive shaft 48 that extends upwardly from the positional sensor 44 along a longitudinal axis of the scan head 40. The drive shaft 48 is rotationally supported within the supporting structure 46 of the scan head 40 by bearings 50 positioned near respective ends of the drive shaft 48. The positional actuator 42 also includes an armature structure 52 that is stationary with respect to the supporting structure 46, and a permanent magnet field structure 54 coupled to the drive shaft 48. When the armature structure 52 is selectively energized, a torque is developed that rotates the drive shaft 48 in a desired rotational direction so that the drive shaft 48 and the field structure 54 form a driven member. The armature structure 52 may also be selectively energized to rotate the drive shaft 48 in increments of less than a full rotation, and/or at different rotational rates during the rotation of the drive shaft 48, as will be described in greater detail below.

The positional actuator 42 further includes a crank member 56 that is coupled to the drive shaft 48, which rotatably couples to a lower, cylindrical-shaped portion of a connecting member 58. The relative position of the crank member 56 with respect to the supporting structure 46 allows adjustment of the mechanical sweeping range of the transducer array assembly 30. An upper end of the connecting member 58 is hingeably coupled to a pivot member 60 that is axially supported on the structure 46 by a pair of bearings 62. The pivot member 60 further supports a cradle 64 that retains the transducer assembly 30. Although not shown in FIG. 2, the cradle 64 may also include electrical contacts so that individual elements in the transducer assembly 30 may transmit and receive ultrasonic signals, as more fully described above. The contacts may further be coupled to a conductive assembly, such as a flex circuit, that is coupled to the processor 12, as shown in FIG. 1. Briefly, and in general terms, rotational motion imparted to the crank member 56 by the drive shaft 48 produces an oscillatory motion in the pivot member 60, which permits the transducer assembly 30 to be moved through a selected scan angle, as will be described in greater detail below. Further, various details of the crank member 56, the connecting member 58 and the pivot member 60 will be shown in greater clarity in another figure.

The positional sensor 44 includes a counter 66 that is stationary with respect to the supporting structure 46, and an encoding disk 68 that is fixedly coupled to the drive shaft 48, so that the encoding disk 68 and the drive shaft 48 rotate in unison. The encoding disk 68 includes a plurality of radially-positioned targets that the counter 66 may detect as the encoding disk 68 rotates through a gap in the counter 66, thus generating a positional signal for the shaft 48. Since the angular position of the array 30 may be correlated with the rotational position of the shaft 48, the encoding disk 68 and the counter 66 therefore cooperatively form a sensor capable of indicating the angular orientation of the array 30. In one particular embodiment, the encoding disk 68 and the counter 66 are configured to detect the rotational position of the drive shaft 48 by optical means. The disk 68 and the counter 66 may also be configured to detect the rotational position of the drive shaft 48 by magnetic means, although still other means for detecting the rotational position of the drive shaft 48 may also be used. In still another particular embodiment, the encoding disk 68 and the counter 66 are configured to have an angular resolution of at least 4000 counts per revolution.

Still referring to FIG. 2, the scan head 40 further includes a cover 70 that is coupled to the supporting structure 46. The cover 70 is formed from a material that is acoustically transparent at ultrasonic frequencies. The cover 70 further partially defines an internal volume 72 that sealably retains an acoustic coupling fluid (not shown) that permits ultrasonic signals to be exchanged between the transducer assembly 30 and the cover 70 by providing a suitable acoustical impedance match. In one aspect, a silicone-based fluid may be used that also provides lubrication to the mechanical elements positioned within the volume 72. A shaft seal 74 is positioned within the supporting structure 46 that surrounds the drive shaft 48 to substantially retain the acoustic coupling fluid within the volume 72. The internal volume 72 further includes an expandable bladder 76 that is positioned below the crank member 56 to permit the fluid retained within the volume 72 to expand as the fluid is heated, thus preventing leakage of the fluid from the volume 72 that may result from excessive fluid pressures developed within the scan head 40.

FIG. 3 is an exploded and partial isometric view of a portion of the positional actuator 42 that will be used to further describe specific details of the actuator 42. For clarity of illustration, the cradle 64 and the transducer assembly 30 of FIG. 2 are not shown. The crank member 56 is fixedly coupled to an upper end of the drive shaft 48 so that the crank member 56 rotates in unison with the drive shaft 48. Accordingly, the crank member 56 is fixedly coupled to the drive shaft 48 by a capscrew 80 that extends through the crank member 56 and is threadably received by the drive shaft 48. Alternatively, the crank member 56 and the drive shaft 48 may be formed as a single integral assembly. The crank member 56 also includes a receiving portion 82 that is angled inwardly towards a rotational axis of the drive shaft 48. The receiving portion 82 rotatably receives a lower cylindrical portion 84 of the connecting member 58, so that the lower portion 84 may freely rotate when positioned within the receiving portion 82. The connecting member 58 also includes an upper hub 86 that includes a bearing recess 88 that extends through the upper hub 86.

Still referring to FIG. 3, the pivot member 60 includes a pair of shafts 65 that are axially received at opposing ends of the member 60. The shafts 65 are retained within the pivot member 60 by means of an interference fit, or by retaining screws, or by still other means. The shafts 65 further receive bearings 62 that form support points between the pivot member 60 and the support structure 46 of FIG. 2. The pivot member 60 also includes a rectangular coupling 94 that is positioned at an approximate midpoint of the pivot member 60 that has a pair of bearings 96 positioned on opposing sides of the coupling 94. The pivot member 60 also includes cradle pads 98 at opposing ends of the pivot member 60 to support the cradle 64 of FIG. 1. A hingeable coupling between the upper hub 86 and the rectangular coupling 94 is formed by a pin 100 that extends through the upper hub 86, the coupling 94 and the bearings 96. A capture screw 90 that is threadably received by the upper hub 86 of the connecting member 58 contacts a surface of the pin 100 so that the pin 100 is retained by the hub 86.

FIG. 4 is a partial isometric view of a portion of the positional actuator 42 that will be used to describe the operation of the of the positional actuator 42 in greater detail. As described earlier in connection with FIG. 2, when the armature 52 is energized, a rotational motion is imparted to the drive shaft 48, which rotates about an axis 102. The drive shaft 48 rotates the crank member 56 so that the receiving portion 82, which retains the lower cylindrical portion 84, also rotates concentrically about the axis 102. Since the upper hub 86 of the connecting member 58 is constrained within the rectangular coupling 94 by the pin 100, the upper hub 86 exerts a torque on the coupling 94 as the crank member 56 rotates so that the pivot member 60 oscillates about an axis 104. Accordingly, the transducer assembly 30 is repetitively moved through a scanning angle 106 as the drive shaft 48 is rotated.

Turning now to FIG. 5, a partial side view of the scan head 40 of FIG. 2 is shown, which will be used to describe scanning angle operating modes according to still another embodiment of the invention. In FIG. 5, the axis 104 (as shown in FIG. 4) projects outwardly from FIG. 5, so that the transducer assembly 30 scans through the scanning angle 106, as described earlier. The scanning angle 106 may be centered about the axis 102, so that the transducer assembly 30 sweeps from the axis 102 to sweep angle limits that correspond to a complete rotation of the drive shaft 48, as shown in FIGS. 2-4. Alternatively, the transducer assembly 30 may be swept through a scanning angle 108 that is less than the scanning angle 106 by controlling the positional actuator 42 (as shown in FIG. 2) to rotate in a first direction less that a full revolution of the drive shaft 48, then rotating the drive shaft 48 in a second direction opposite to the first direction. Accordingly, scanning angles that are less than the scanning angle 106, which is the maximum obtainable scanning angle, may be conveniently obtained.

Still referring to FIG. 5, the positional actuator 42 may also be controlled to sweep the transducer assembly 30 about an angle that is centered on another axis 110 that is oriented at an angle with respect to the axis 102 so that the transducer assembly 30 may scan into anatomical regions that cannot be adequately scanned when the transducer assembly 30 is scanned through angles centered about the axis 102. For example, in performing an ultrasound scan in an upper abdominal or thoracic region, it is often difficult to properly position a scan head so that interfering reflections from ribs or other tissues is avoided. The ability to scan about an axis 110 that is not aligned with a longitudinal axis of the support structure 46 of the scan head is therefore regarded as particularly advantageous.

FIG. 6 is a graph that illustrates the angular position 120 of the transducer assembly 30 as it sweeps through the scanning angle 106 shown in FIG. 5. The angular position 120 is sinusoidal when the drive shaft 48 is rotated at a constant angular speed co. Accordingly, the transducer assembly 30 exhibits a time-varying scanning rate 122 as the assembly 30 is swept through the scanning angle 106. Since the scanning rate 122 varies as shown in FIG. 6, the transducer assembly 30 is moved through a sweep angle interval 124 at a relatively slow rate, and is moved at a relatively high rate when the transducer assembly 30 moves through the sweep angle interval 126. Accordingly, scan lines associated with ultrasound emissions from the assembly 30 will not be spaced at regular intervals when the processor 12 (as shown in FIG. 1) emits pulses of ultrasound energy at a constant rate. As a consequence, the frame rate will also be non-uniform as the transducer assembly 30 is moved through the scanning angle 106. One difficulty stemming from a non-constant frame rate is that the resulting images may exhibit significant differences in elevation resolution, thus making diagnostic interpretation more difficult.

FIG. 7 is a graph that illustrates a method for controlling a scanning rate for a scan head according to yet another embodiment of the invention. FIG. 7 shows the variation of the scanning rate for the scan head 40 of FIG. 2. For reference purposes, the scanning rate 122, which corresponds to a constant angular speed co of the drive shaft 48 of FIG. 2 is shown. In one particular embodiment, a scanning rate 130 having a relatively constant value over a substantial portion of the scanning angle 106 may be obtained by suitably controlling the armature structure 52 (as shown in FIG. 2) to impart a non-constant angular rotation rate to the drive shaft 48. Since the scanning rate 130 is relatively constant, the lateral distance between adjacent scan lines becomes more uniform and resolvable, which permits the formation of images having higher efficiency and lower distortion.

Still referring to FIG. 7, other scanning rates may be obtained by similarly controlling the armature structure 52. In another particular embodiment, a scanning rate 132 is obtained by controlling the armature structure 52 to a first value to obtain a relatively constant rotational rate for the drive shaft 42 for a first scan angle portion 134, then controlling the armature structure 52 to a second value to obtain a relatively constant scanning rate for a second scan angle portion 136, following which the armature structure 52 is again controlled to the first value during a third scan angle portion to obtain a relatively constant rotational rate for the drive shaft 42. In still another particular embodiment, the first scan angle portion 134 is approximately about, or less than about 18 degrees.

The above description of illustrated embodiments of the invention is not intended to be exhaustive or to limit the invention to the precise form disclosed. While specific embodiment of, and examples of, the invention are described in the foregoing for illustrative purposes, various equivalent modifications are possible within the scope of the invention, as those skilled within the relevant art will recognize. Moreover, the various embodiments described above can be combined to provide further embodiments. Accordingly, the invention is not limited by the disclosure, but instead the scope of the invention is to be determined entirely by the following claims.

1. An ultrasound imaging system 10, comprising:
a processing system 12 configured to generate ultrasound energy and to detect signals at ultrasound frequencies; and
an ultrasound scan head 40 electrically coupled to the processing system 12, the assembly 30 including an ultrasound transducer array operatively coupled to a positional actuator 42 having a driven member that rotates about a first axis to pivot the array about a second axis substantially perpendicular to the first axis.

2. The imaging system 10 of claim 1, wherein the ultrasound scan head 40 includes a positional sensor 44 coupled to the driven member that is configured to detect a rotational position of the driven member.

3. The imaging system 10 of claim 2, wherein the processing system 12 further includes a controller 26 electrically coupled to the positional actuator 32 to transmit positioning signals to the actuator 32, and to receive positional signals from the positional sensor 34.

4. The imaging system 10 of claim 1, wherein the ultrasound transducer array 30 comprises a planar arrangement of ultrasound transducer elements.

5. The imaging system 10 of claim 1, wherein the ultrasound transducer array 30 comprises a linear arrangement of ultrasound transducer elements that is curved along a length of the array.

6. The imaging system 10 of claim 3, further comprising a display coupled to the controller, the display operable to visually display ultrasound images generated by the processor 12.

7. An ultrasound scan head 40 for ultrasound imaging, comprising:
an ultrasound transducer array 30 having a plurality of transducer elements for transmitting acoustic energy in response to an applied electrical signal and transducing returned acoustic energy into electrical signals;
a positional actuator 42 having a driven member configured to be rotated about a first rotational axis and coupled to a pivot member that supports the array 30, the pivot member being configured to rotate about a second rotational axis substantially perpendicular to the first axis; and
a positional sensor 44 coupled to the positional actuator 42 and operable to sense a rotational position associated with the positional actuator 42.

8. The ultrasound scan head 40 of claim 7, wherein the positional actuator 42 includes a permanent magnet field structure 54 coupled to a drive shaft 48, and an armature structure 52 that is stationary with respect to the drive shaft 48.

9. The ultrasound scan head 40 of claim 7, wherein the positional actuator 42 includes a crank member 56 coupled to a drive shaft 48, the crank member 56 having a receiving portion angled inwardly towards the first rotational axis, and the pivot member 60 includes a coupling, the crank member 56 being coupled to the pivot member 60 by a connecting member 58 that is rotatably received by the receiving portion at one end, and hingeably received by the coupling at an opposing end.

10. The ultrasound scan head 40 of claim 9, comprising:
a cover 70 positioned proximate to the array that at least partially defines an internal volume 72 that contains the array 30, the internal volume 72 sealably containing a volume of an acoustic coupling fluid.

11. The ultrasound scan head 40 of claim 10, wherein the internal volume 72 includes an expandable bladder 76 that adjusts to variations in the volume 72 of the acoustic coupling fluid.

12. The ultrasound scan head 40 of claim 7, wherein the positional sensor 44 includes a sensor capable of detecting an angular position of the driven member by optical means.

13. The ultrasound scan head 40 of claim 7, wherein the positional sensor 44 includes a sensor capable of detecting an angular position of the driven member by magnetic means.

14. The ultrasound scan head 40 of claim 7, wherein the positional sensor 44 includes a counter 66 having an angular resolution of at least-1000 counts per revolution.

15. In a scan head 40 having a driven member rotatable about a first axis and coupled to an ultrasound array 30 rotatable about a second axis, a method for three-dimensional imaging a portion of a body, comprising:
controlling the rotation of the driven member over a predetermined rotational interval to provide approximately constant rotation of the array 30; and
acquiring ultrasound data along a plurality of mutually spaced-apart scan lines.

16. The method of claim 15, wherein controlling the rotation of the driven member further comprises varying the sweeping range of the transducer array 30 to improve scan efficiency with a continuous variable speed rotation.

17. The method of claim 15, wherein acquiring ultrasound images further comprises acquiring the images along scan lines that are approximately equally spaced apart.

18. The method of claim 15, wherein controlling the rotation of the driven member further comprises maintaining the rotation of the driven member at a constant rotational value for a first rotational interval; and controlling the rotation of the driven member to provide an approximately constant rotation of the array for a second rotational interval.

19. The method of claim 15, wherein acquiring ultrasound images further comprises processing the data to develop an ultrasound image.

20. The method of claim 15, further comprising:
repetitively sweeping the array across the body portion; and
obtaining a data image set corresponding to each successive sweep.
